(19) United States
(12) Patent Application Publication
(10) Pub. No.: US 2008/0004899 A1
(43) Pub. Date: Jan. 3, 2008

(54) System and method for representing a patient data

(76) Inventor: John H. Braxton, Cape Elizabeth, ME (US)

Correspondence Address: KEVIN FARRELL;PIERCE ATWOOD, ONE NEW HAMPSHIRE AVENUE, PORTSMOUTH, NH, 03801, US

(21) Appl. No.: 11/477,694

(22) Filed: Jun. 29, 2006

(51) Int. Cl. G06Q 10/00; A61B 5/00

(52) U.S. Cl. 705/2; 600/300

(57) ABSTRACT

The present invention includes a system and method for representing patient data, preferably in a clinical context. The system of the present invention generally includes a central computer having a database, wherein the database includes clinical data for a number of patients. The computer further includes a representation unit that is configured to select actionable data for each patient from that patient's clinical data. The actionable data is then processed and formatted such that it can be represented and readily conveyed to a user, such as for example an attending health care professional. The method of the present invention generally includes the steps of inputting clinical data regarding a patient into a selection algorithm, selecting actionable data from the clinical data, and representing a status of the patient in response to the actionable data. The representation of the patient status may take the form of qualitative and quantitative presentations that are complimentary and non-exclusive in nature.

### TECHNICAL FIELD

The present invention relates generally to the field of information technology, and more particularly to systems and associated methods usable in the medical arts for the treatment of patients.

### BACKGROUND AND SUMMARY OF THE INVENTION

Contemporary medical practice is primarily a human endeavor, requiring the coordination and efforts of countless medical professionals in order to treat a growing number of patients. An example of an essential relationship exists between an attending physician and the one or more medical interns that supervise the treatment of patients in a hospital context. Typically, a medical intern is a recent medical school graduate that is charged with monitoring the status of a number of patients in a hospital while the attending physician is the professional that is ultimately responsible for the diagnosis and treatment of any disease processes that affect the patients. In the United States, for example, this division of labor has worked for well over a century, and resulted in some of the best-trained physicians in the world that deliver some of the most cutting edge treatment in history.

In spite of this historical success, the use of medical interns to monitor patients and collect patient data does have some drawbacks. First, as the populations of the most medically advanced countries age, the number of patients that require treatment each year is outpacing the number of available medical interns that are available for patient care. Secondly, the amount of labor required by each medical intern in performing his or her duties is also increasing with the increased number of patients, the advent of new treatments for old disease processes, and the unfortunate discoveries of new diseases and maladies that affect the patients. As the number of patients continues to grow, the corresponding strain on the medical interns poses a serious problem that may inadvertently lead to mistreatment due to the overwhelming amount of patient data that requires collection and analysis. Moreover, exhaustion of the existing medical interns has spurred cries for relief in their work schedules, as in the recent past medical interns were well-known for spending one hundred hours or more at the hospital each week. Unfortunately, demographic concerns and rising health-care costs have combined in such a manner that the contemporary practice appears unfit to continue treating patients according to the best practices of the medical profession.

As such, there is a need in the art for a system and method of representing patient data that combines the traditional successes of diagnosis and treatment in the medical profession while reducing the overall labor costs associated with the contemporary practice. Moreover, there is a need in the art for a system and method of representing patient data that is adapted to make the medical practice more efficient, by distinguishing between that patient data that is relevant to the treatment of the existing disease process. In particular, there is a tendency for medical interns to conflate that patient data which is actionable with that patient data which is non-actionable, thus putting the burden on the attending physician of distinguishing between the two in determining the proper course of therapy for the patient. Finally, there is a need in the art for such a system and a method that can readily perform the essential tasks of a medical intern using developments in the fields of telecommunications and information technology that are best suited for integration with the existing computing and data storage capabilities of medical facilities.

Accordingly, the present invention provides for such a system and method of representing patient data. The system of the present invention generally includes a central computer having a database, wherein the database includes clinical data for a number of patients. The computer further includes a representation unit that is configured to select actionable data for each patient from that patient's clinical data. The actionable data is then processed and formatted such that it can be represented and readily conveyed to a user, such as for example an attending health care professional. In some of the exemplary embodiments discussed below, the actionable data is formatted according to both qualitative and quantitative norms, thus providing an attending physician with a quick reference point for determining the status of the patient relative to the normalized course of therapy.

The present invention further includes a method of representing patient data. The method of the present invention generally includes the steps of inputting clinical data regarding a patient into a selection algorithm, selecting actionable data from the clinical data, and representing a status of the patient in response to the actionable data. The representation of the patient status may take the form of qualitative and quantitative presentations that allow an attending physician to make a quick judgment concerning the status of the patient relative to the normalized course of therapy. In some of the exemplary embodiments discussed below, the method of the present invention is practiced by one or more software programs that are operable on the system of the present invention.

Although briefly summarized here, the preferred embodiments of the present invention are described more fully below with reference to the following Figures.

### BRIEF DESCRIPTION OF THE FIGURES

FIG. 1 is a flow chart depicting a method for representing patient data in accordance with a preferred embodiment of the present invention.

FIG. 2 is a flow chart depicting a method for representing patient data in accordance with another preferred embodiment of the present invention.

FIG. 3 is a flow chart depicting a method for representing patient data in accordance with another preferred embodiment of the present invention.

FIG. 4 is a flow chart depicting a method for representing patient data in accordance with another preferred embodiment of the present invention.

FIG. 5 is a schematic block diagram of a system for representing patient data in accordance with a preferred embodiment of the present invention.

FIG. 6 is a view of a user display representing patient data in accordance with a preferred embodiment of the present invention.

FIG. 7 is a view of a user display representing patient data in accordance with another preferred embodiment of the present invention.

FIG. 8 is a view of a user display representing patient data in accordance with another preferred embodiment of the present invention.

FIG. 9 is a view of a user display representing patient data in accordance with another preferred embodiment of the present invention.

FIG. 10 is a view of a user display representing patient data in accordance with another preferred embodiment of the present invention.

FIG. 11 is a view of a user display representing patient data in accordance with another preferred embodiment of the present invention.

FIG. 12 is a view of a user display representing patient data in accordance with another preferred embodiment of the present invention.

### DESCRIPTION OF THE PREFERRED EMBODIMENTS

The following description of various preferred embodiments of the invention is not intended to limit the invention to these preferred embodiments, but rather to enable any person skilled in the art of medical imaging to make and use this invention.

As such, the present invention includes a system and a method for representing patient data in a clinical context. As used herein, the term patient should be understood to include both human and animal patients, as the present invention is usable in both the practice of medicine and veterinary medicine. The method the present invention generally includes the steps of receiving clinical data regarding a patient, selecting actionable data from the clinical data, and representing a status of the patient in response to the actionable data. The system of the present invention includes a central computer including a database of patient data and a representation unit coupled to the database of patient data. The representation unit includes a selection algorithm adapted to select between actionable and non-actionable data and a presentation unit adapted to format the actionable data into a presentable format. In preferred embodiments, the system further includes a mobile device communicable with the central computer, the mobile computer including a display for displaying actionable data receivable in the presentable format from the central computer.

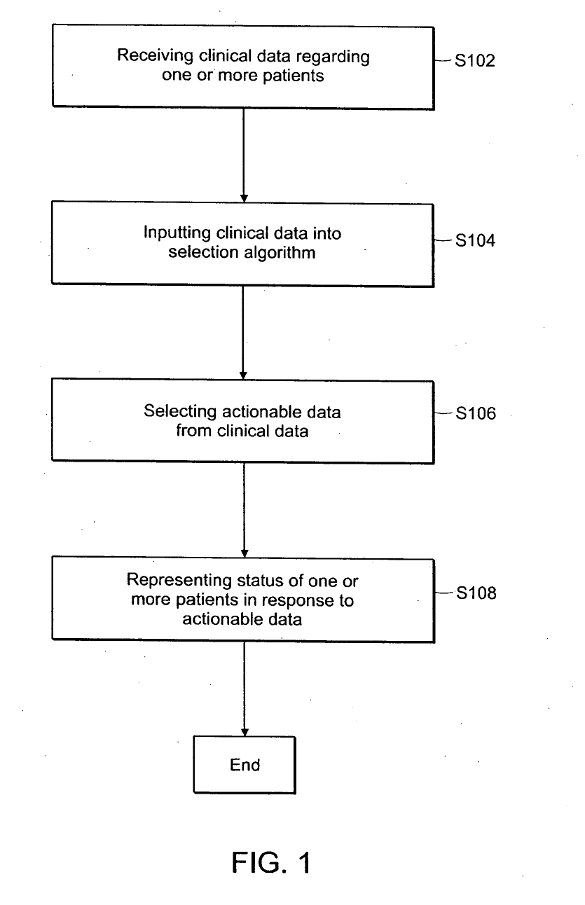

As shown in the flowchart of FIG. 1, step S102 of the preferred method includes receiving clinical data regarding one or more patients. Clinical data, as used herein, includes any and all data related to the health of a patient, such as for example a human or animal patient. Clinical data should be understood in its broadest sense, incorporating all aspects of a patient's condition, including past and present data regarding the patient's medical, psychological, physiological, anatomical histories and conditions. Typically, the step of receiving clinical data includes conducting an interview of the patient, the patient's representatives, or other observation of the patient, including any third-party information regarding an accident, prior medical history, pre-existing conditions, allergies, prescription medications, and the like. Preferably, the clinical data is then input into one or more computers according to methods known in the art.

In step S104, the preferred method recites the step of inputting the clinical data into a selection algorithm. The process of inputting the clinical data into the selection algorithm can be accomplished by automated or manual means. Preferably, the clinical data is assembled by an intern, nurse or technician during the patient intake procedure as described above. Following receipt of the clinical data, the preferred method then automatically inputs the clinical data into the selection algorithm of the present invention. As the care of any patient is typically an extended process, the clinical data is subject to augmentation, amendment and alteration over a time period. As any changes are made to a patient's clinical data, those changes are preferably input into the selection algorithm through automated means. As noted above, the clinical data is typically entered and stored on one or more computers. Accordingly, the preferred selection algorithm is a software application that is operable on one or more computers that are either identical to, or connected to, the one or more computers that receive the clinical data.

In step S106, the preferred method recites the step of selecting actionable data from the clinical data. Step S106 is preferably performed by the selection algorithm, which is preferably a software application operable on one or more computers as noted above. The selection algorithm is adapted to select between actionable data and non-actionable data from the clinical data. As used herein, the term actionable data includes a subset of the clinical data that is directly or indirectly relevant to the patient's care, including without limitation data regarding any injuries, maladies or disease processes that are or may reasonably be afflicting the patient. The term non-actionable data should be construed to include all clinical data that is not actionable data.

In step S108, the preferred method recites the step of representing a status of the one or more patients in response to the actionable data. Preferably, the representation includes a diagram, chart, graph or other readily communicable measurement of the patient's status. For example, the status of the patient may be visually represented on a display portion of a computing device, wherein the representation may take the form of a graph, chart or diagram that readily conveys the patient's status to a skilled viewer. Alternatively, other means for representing the patient's status, such as aural means, may be utilized in step S108 of the preferred method.

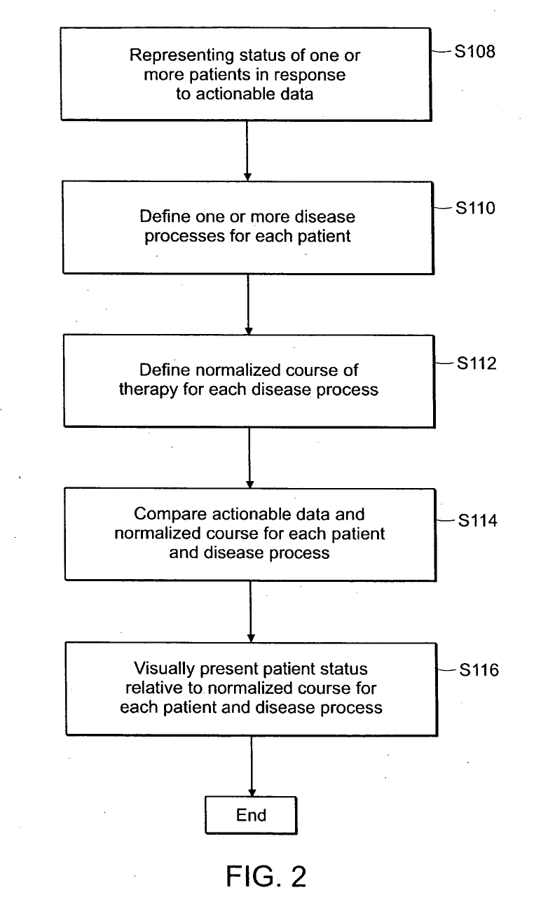

In alternative embodiments, the preferred method of the present invention includes additional steps for representing the status of the patient. As shown in the flow chart of FIG. 2, the preferred method includes step S110, which recites defining one or more disease processes for each patient. As used herein, the term disease process includes any known or reasonably well-known process that adversely affects a human or animal patient, not limited necessarily to diseases but rather including ancillary afflictions, conditions and disorders as well. For example, the term disease process includes any coronary diseases as well as the effects thereof, such as palpitations, arrhythmias, cardiac arrests and any other cardio or cardiopulmonary affliction or disorder. Accordingly, each patient may have more than one disease process at any given time, such as a stroke and cardiac arrest, each of which include numerous adverse effects that are also includable within the scope of step S110 of the present invention.

Another aspect of the preferred method is included in step S112, which recites defining a normalized course of therapy for each disease process. As noted above, the term disease process includes any known or reasonably well-known process that adversely affects a human or animal patient. The normalized course of therapy therefore is any known or reasonably well-known course of treatment that is adapted to cure, mitigate or otherwise alter the disease process. For example, normalized courses of therapy may include surgery and other interventional means, radiological treatments, pharmaceutical treatments, psychological or psychiatric treatments, as well as occupational or physical therapy. As noted above, a patient may have more than one disease process at the same time, and as such each patient may also be undergoing more than one normalized course of therapy for each disease process. Likewise, each individual disease process may include more than one normalized course of therapy, for example one course of therapy for treatment and mitigation of symptoms and another course of therapy to address the underlying cause of the disease process.

In step S114, the preferred method recites the step of comparing the actionable data and normalized course of therapy for each patient and for each disease process. As noted above, the actionable data includes a subset of the clinical data that is directly or indirectly relevant to the patient

s care, including without limitation data regarding any injuries, maladies or disease processes that are or may reasonably be afflicting the patient. Therefore, comparison of the actionable data and the normalized course of therapy for each patient results in a measurement or determination of the patient's progress in his or her treatment. If the actionable data is sufficiently commensurate with the normalized course of therapy for a patient, then the patient is making substantial progress in his or her course of therapy. Conversely, if the actionable data is sufficiently divergent from the normalized course of treatment, then the patient is not making substantial progress in his or her course of therapy.

Step S116 recites visually presenting the patient status relative to the normalized course of treatment for each patient and for each disease process affecting the patient. As noted above, comparison of the actionable data and the normalized course of therapy for each patient results in a measurement or determination of the patient's progress in his or her treatment. The measurement or determination resulting from step S114 is then presented in step S116. Preferably, this presentation is visual in nature such that a user can readily determine a patient's status with regard to the one or more disease processes that are affecting him or her. Suitable means for visually presenting the patient status include displays or monitors that are coupled to or integrated into computers, which may be portable or affixed near one or more patients. In particular, graphs, charts and diagrams are preferred methods for visually presenting the patient's status as a user can quickly assimilate the necessary information to make an informed judgment regarding the patient's status.

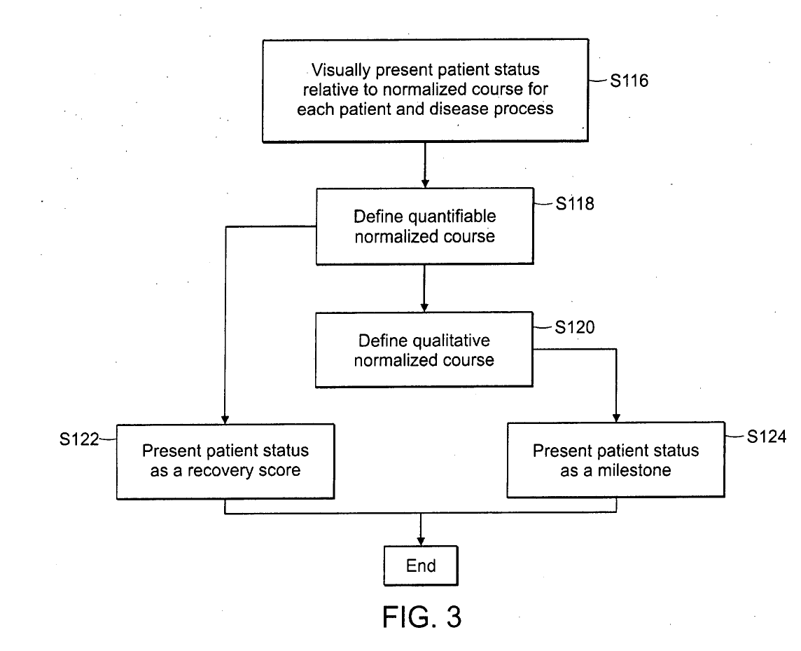

In one alternative preferred embodiment, the normalized course of therapy is defined as a quantifiable metric. In another alternative preferred embodiment, the normalized course is defined as a qualitative metric. By way of example, FIG. 3 is a flow chart depicting these alternative preferred embodiments, which are complimentary, and not exclusive, methods for presenting the status of a patient. From step S116, described above, the method proceeds to step S118, which recites defining a quantifiable normalized course of treatment. An example quantitative normalized course of treatment is a recovery score, which is presented in step S122 of the preferred method of the present invention. In step S120, the method recites defining a qualitative normalized course of treatment. An example qualitative normalized course of treatment is a milestone, which is presented in step S124 of the preferred method of the present invention. The recovery score and milestone tools are described in further detail below.

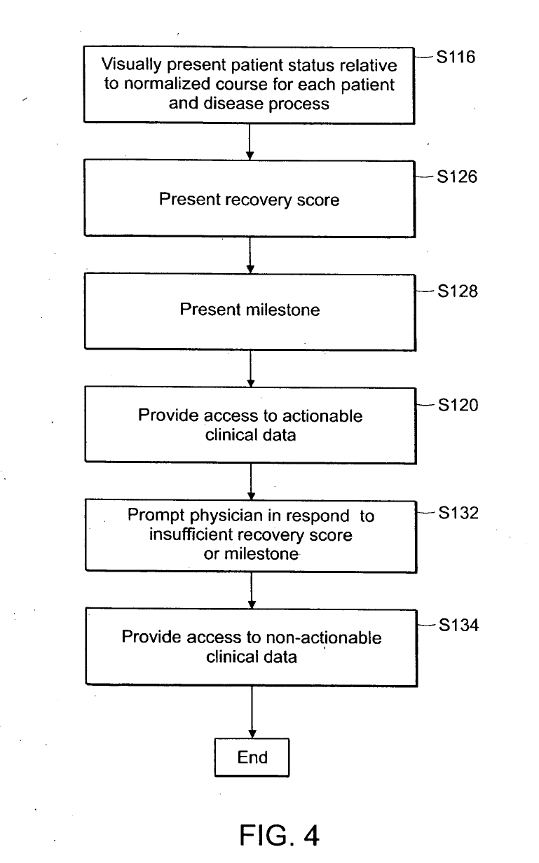

In another aspect of the present invention, the preferred method provides for prompting a physician, nurse or other skilled professional as to the status of the patient. As shown in the flowchart of FIG. 4, the preferred method includes step S116 that recites visually presenting the patient status relative to the normalized course for each patient and disease process affecting the patient. The visual presentation, as discussed above, may take the form of a recovery score in step S126 or a milestone in step S128, which are quantitative and qualitative means for representing the patient data as described above. In step S130, the preferred method provides access to the actionable clinical data, as defined above. Thus, as a user is apprised of the patient's status through the visual presentation of that status, the user may elect to review the actionable clinical data in its raw format, i.e. in a format that has not been quantified or qualified according to the prior steps of the preferred method.

In the event that the patient is not making sufficient progress in his or her course of treatment, the preferred method prompts the user as to this insufficiency. In step S132, the method recites the step of prompting the physician in response to an insufficient recovery score or an insufficient milestone. The prompting may take the form of a visual or aural cue or signal to a user that the recovery score or milestone is not being met, or that the patient's quantitative or qualitative recovery is not within a predetermined threshold of the normalized course of therapy. The threshold limits of the normalized course of therapy may be determined on a case by case basis by the user, such as for example a physician or other qualified medical professional. Alternatively, the threshold limits of the normalized course of therapy may be defined according to the disease process, with further modifications being made in consideration of the patient's age; medical history and any other mitigating or aggravating circumstances. For example, a suitable recovery from a cardiac arrest may be different for a 45-year old than it would be for a 75-year old, and thus the present invention allows for modification of the threshold limits of the normalized course of therapy with these variations in mind.

The method also provides access to non-actionable clinical data in step S134. This step may be performed in response to an insufficient recovery score or milestone for a patient. Alternatively, the user may access the non-actionable clinical data in order to update, revise or otherwise modify the threshold limits of the normalized course of therapy. In either event, the present invention provides the non-actionable clinical data to ensure that the user has access to the complete clinical data for the patient, which is of particular relevance if the patient's recovery is not progressing as well as hoped.

The preferred method of the present invention is preferably performed by a system for representing patient data described further herein. The preferred system of the present invention includes a central computer having a database of patient data and a representation unit coupled to the database of patient data. The preferred representation unit includes a selection algorithm adapted to select between actionable and non-actionable data and a presentation unit adapted to format the actionable data into a presentable format. The system further includes a mobile device communicable with the central computer. The preferred mobile device includes a display for displaying actionable data receivable in the presentable format from the central computer.

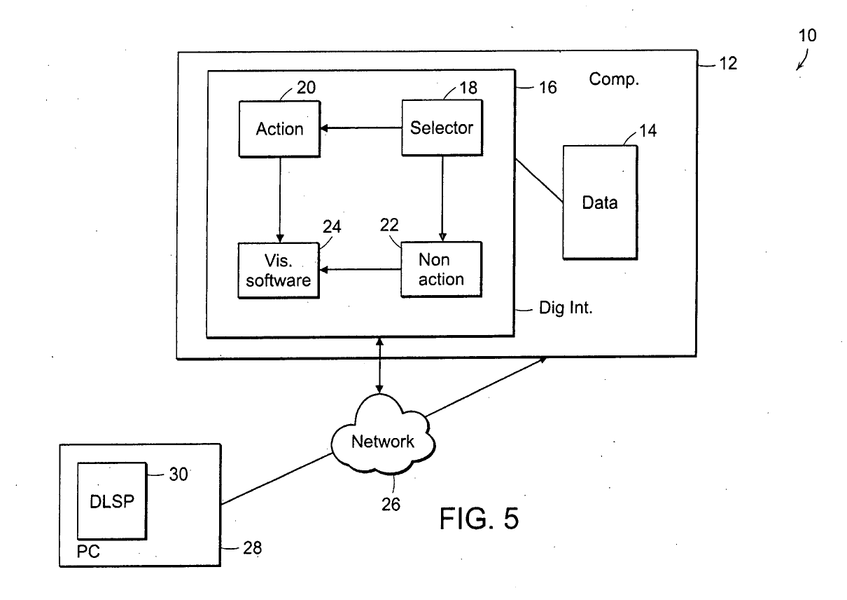

As shown in FIG. 5, an embodiment of the system 10 of the present invention includes a computer 12 having a database 14 stored thereon. The computer 12 is preferably a mainframe or other large computer of the type found in hospitals and clinics having sufficient storage and processing capabilities for managing a large volume of patient data. Alternatively, in smaller applications, the computer 12 may be a desktop or personal computer of the type known in the art that is configured with sufficient storage and processing capabilities for managing a large volume of patient data. The database 14 is preferably located within the computer 12 and configured for storing, at least in part, the relevant clinical data for one or more patients. Alternatively, the database 14 may be located remotely from the computer 12 and in communication therewith through a network or other communication means. For example, the database 14 may be a large backup drive that is remotely disposed from the principal computer 12 in order to disperse and protect the clinical data of the one or more patients.

In an alternative embodiment, the database 14 further includes data regarding one or more disease processes that have been diagnosed for each patient. As noted above, the term disease process includes any known or reasonably well-known process that adversely affects a human or animal patient, not limited necessarily to diseases but rather including ancillary afflictions, conditions and disorders as well. Example disease processes include any coronary diseases as well as the effects thereof, such as palpitations, arrhythmias, cardiac arrests and any other cardio or cardiopulmonary affliction or disorder. Each patient may have more than one disease process at any given time, such as a stroke and cardiac arrest, each of which include numerous adverse effects that are also includable within the scope of the term disease process as used herein.

In another alternative embodiment, the database 14 includes data regarding a normalized course of therapy for each disease process and each patient. As noted above, the term disease process includes any known or reasonably well-known process that adversely affects a human or animal patient. The normalized course of therapy therefore is any known or reasonably well-known course of treatment that is adapted to cure, mitigate or otherwise alter the disease process. For example, normalized courses of therapy may include surgery and other interventional means, radiological treatments, pharmaceutical treatments, psychological or psychiatric treatments, as well as occupational or physical therapy. As noted above, a patient may have more than one disease process at the same time, and as such each patient may also be undergoing more than one normalized course of therapy for each disease process. Likewise, each individual disease process may include more than one normalized course of therapy, for example one course of therapy for treatment and mitigation of symptoms and another course of therapy to address the underlying cause of the disease process.

The computer 12 further includes a representation unit 16 that is preferably coupled to the database 14. The preferred representation unit 16 functions to retrieve and process patient data, including clinical data, such that said data is presentable to a user in conjunction with the system and method of the present invention. The preferred representation unit 16 includes a software module or program that is operable on a computer such as the type noted above. Alternatively, the representation unit may include a combination of hardware and software components that are operable on or in conjunction with a computer of the type noted above. In particular, the representation unit 16 includes means for accessing patient data, including clinical data for one or more patients, that is stored or storable on the database 14. Moreover, as described in more detail below, the representation unit 16 is communicable or adapted to communicate with one or more mobile devices that may be used in conjunction with the system and method of the present invention.

The preferred representation unit 16 further includes a selection algorithm 18. The preferred selection algorithm 18 is embodied in software, hardware, or a combination of software and hardware that is disposed within or otherwise coupled via a network to the representation unit 16 and the computer 12. The selection algorithm 18 functions to receive the clinical data of one or more patients and divide the clinical data into an actionable data set 20 and a non-actionable data set 22. As noted above, the term actionable data includes a subset of the clinical data that is directly or indirectly relevant to the patient's care, including without limitation data regarding any injuries, maladies or disease processes that are or may reasonably be afflicting the patient. The term non-actionable data should be construed to include all clinical data that is not actionable data.

In an alternative embodiment, the selection algorithm is adapted to select between actionable and non-actionable data for each patient in response to the disease process and the normalized course of therapy associated with that disease process. The actionable data includes a subset of the clinical data that is directly or indirectly relevant to the patient's care, including without limitation data regarding any injuries, maladies or disease processes that are or may reasonably be afflicting the patient. Therefore, selection of the actionable data is best based on the one or more disease processes that are affecting the patient. Similarly, the selection of the actionable data is affected by the normalized course of therapy for each patient, which results in a measurement or determination of the patient's progress in his or her treatment. If the actionable data is sufficiently commensurate with the normalized course of therapy for a patient, then the patient is making substantial progress in his or her course of therapy. Conversely, if the actionable data is sufficiently divergent from the normalized course of treatment, then the patient is not making substantial progress in his or her course of therapy.

The preferred representation unit 16 further includes a presentation unit 24 that is coupled to the selection algorithm 18. The presentation unit 24 functions to format the actionable data into a presentable format, such as for example a chart, graph or other visual format that is readily conveyed to a user. Preferably, the presentation unit 24 is embodied in software, hardware, or a combination of software and hardware that is contained in or coupled to the representation unit 16 and the computer 12. In alternative embodiments, the presentation unit 24 may be adapted to format the actionable data into a presentable format, or both the actionable and non-actionable data into a presentable format. Most preferably, the presentation unit 24 is adapted to format the actionable data into a quantitative and a qualitative measure of a patient's status in response to the actionable data in response to the disease process and the normalized course of therapy for each patient.

In the event that the patient is not making sufficient progress in his or her course of treatment, the preferred presentation unit 24 prompts the user of the mobile device 28 as to this insufficiency. In particular, the prompting may take the form of a visual or aural cue or signal to a user that the patient's quantitative or qualitative recovery is not within a predetermined threshold of the normalized course of therapy. The threshold limits of the normalized course of therapy may be determined on a case by case basis by the user, such as for example a physician or other qualified medical professional. Alternatively, the threshold limits of the normalized course of therapy may be defined according to the disease process, with further modifications being made in consideration of the patient's age, medical history and any other mitigating or aggravating circumstances. As noted above, a suitable recovery from a cardiac arrest may be different for a 45-year old than it would be for a 75-year old, and thus the present invention allows for modification of the threshold limits of the normalized course of therapy with these variations in mind.

The preferred system 10 further includes a mobile device 28 that is coupled to the computer 12 through a network 26 of the type known in the art of computer networking. The preferred mobile device 28 functions to receive from the computer 12 at least the actionable data as formatted into the presentable format described above. To that end, the preferred mobile device 28 includes a display 30 and the associated processing hardware and software in order to display the actionable data in the presentable format to a user. The mobile device 28 may be any device having the requisite data processing and communications capabilities that is not identical to the computer 12. Example mobile devices 28 include stationary personal computers located in patient rooms, movable laptop computers, personal digital assistants, cellular or digital mobile telephones, mobile electronic-mail devices, and any other device that functions to receive and display the actionable data in the presentable format. The mobile device 28 may be coupled to the computer 12 through wired or wireless means, according to known protocols known in the art of computer networking. Preferably, the network 26 includes the requisite security protocols to ensure the confidentiality and protection of the clinical data 14 stored on the computer 12. In a typical patient care setting, more than one mobile devices 28 can be used with the computer 12 through the network 26.

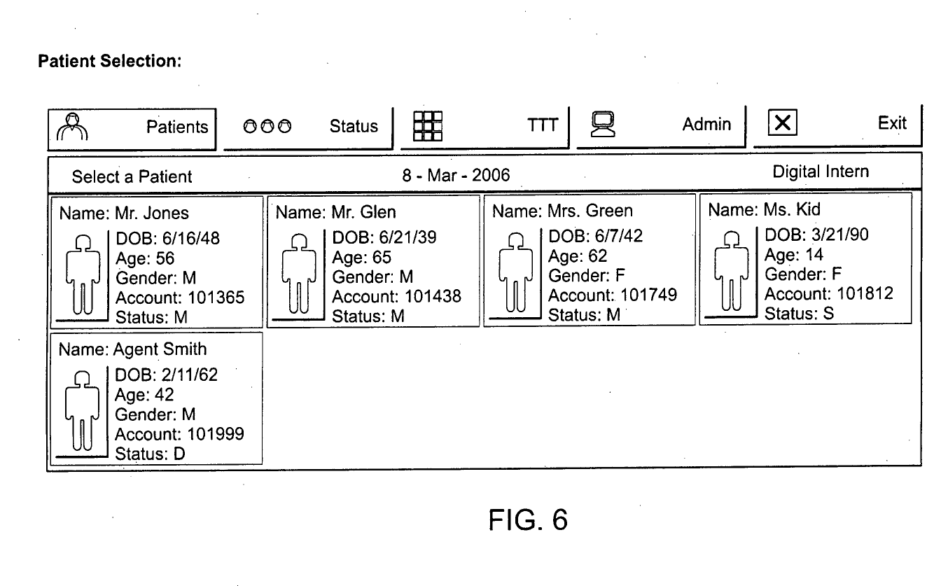

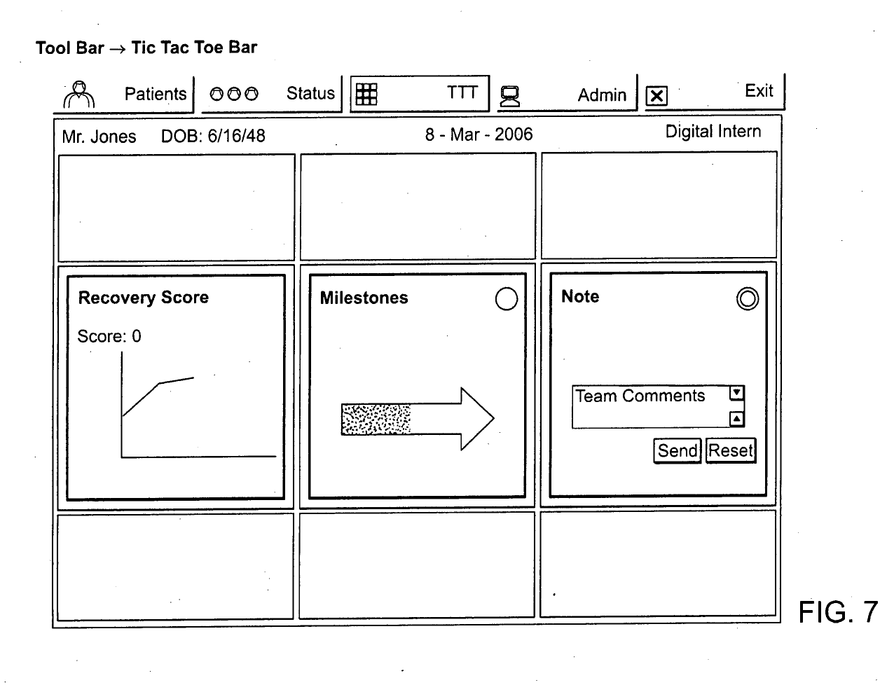

FIG. 6 is illustrative of a sample display 30 that visually conveys the status of one or more patients to a user. As shown, the display 30 displays information for a plurality of patients, as well as a menu from which a user may select to examine a patient's status. As noted above, a preferred means for representing patient status includes both a quantitative and a qualitative measure of the patient's progress as compared to the normalized course of therapy. The alternative preferred means for representing patient status are complimentary, and not necessarily exclusive. An example quantitative normalized course of treatment is a recovery score, which is selectable from a menu provided on the display as shown in FIG. 7. An example qualitative normalized course of treatment is a milestone, which is also selectable from the menu provided on the display as shown in FIG. 7.

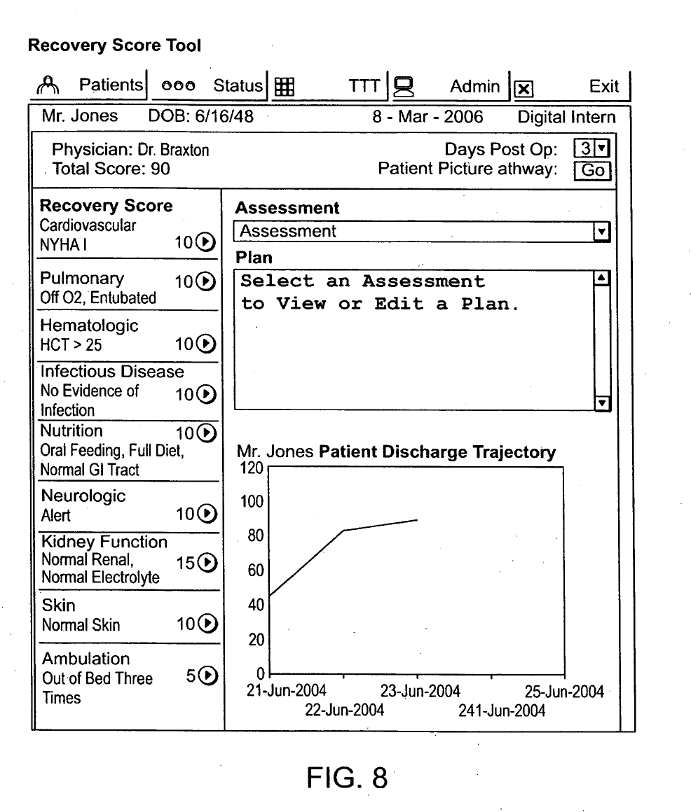

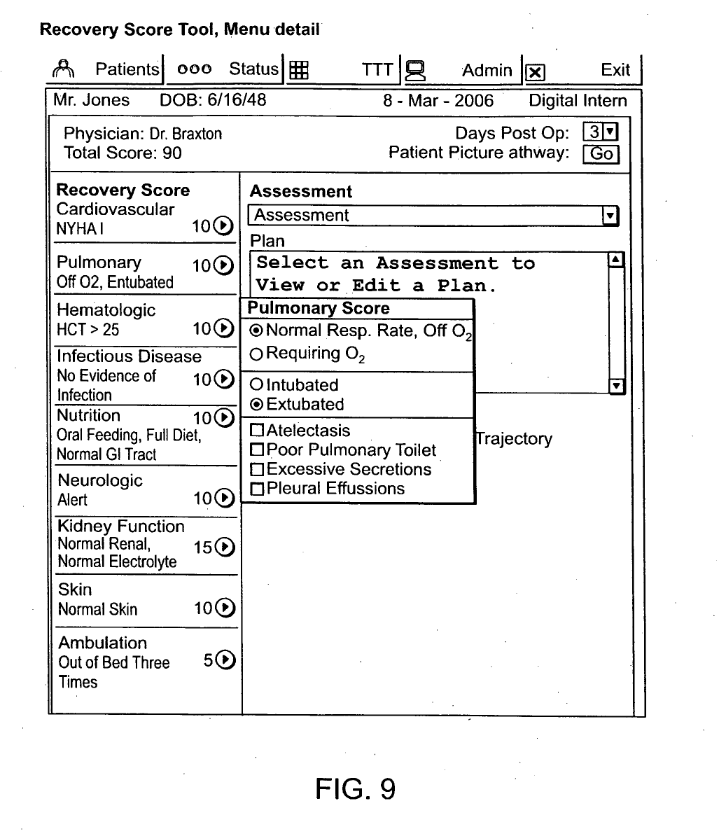

The recovery score tool is shown in further detail in FIG. 8, which is an example of the display 30 of the present invention. As shown, the recovery score includes a numerical indicator of a patient's total score, i.e. ninety, as well as the individual scores for a patient's actionable data. Thus, for the sample patient Mr. Jones, the recovery score for his cardiovascular treatment is a ten, while his kidney function scores a fifteen. Each of the individual scores are summed to form the aggregate total score, which is a quantitative measure of the patient's status as compared to the normalized course of therapy for the one or more disease processes affecting the patient. As shown in FIG. 9, each of the individual scores for the patient's actionable data can be further examined by the user by selecting on the relevant score. Thus, the pulmonary score for Mr. Jones is a ten, which indicates that the patient has a normal respiratory rate and does not require external oxygen and further that the patient has been extubated. If, for example, Mr. Jones required external oxygen, then the pulmonary score would be less than ten and the total score would be less than ninety, indicating that Mr. Jones required extra treatment or care in order to be released from the system.

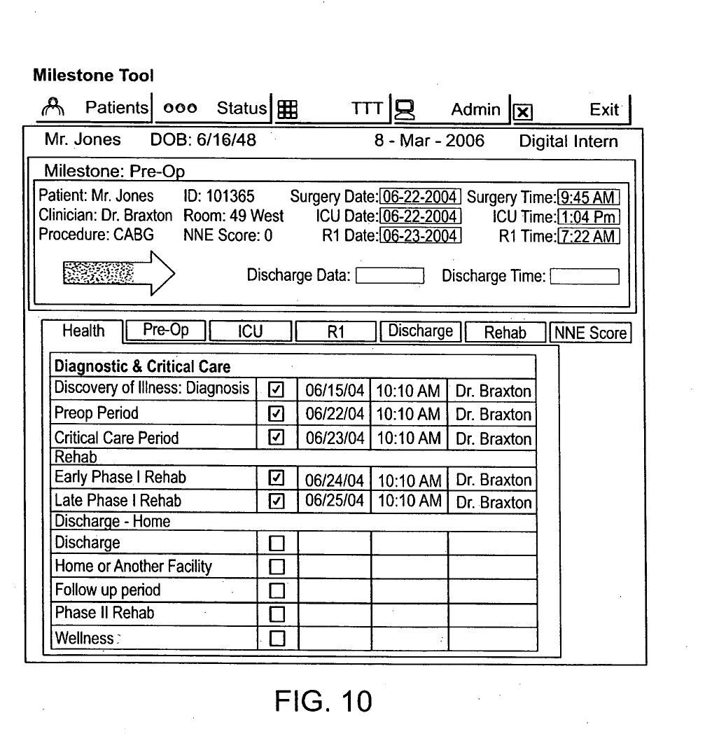

The milestone tool is shown in greater detail in FIG. 10, which is an example of the display 30 of the system 10 of the present invention. As noted above, the milestone tool is a preferred means for representing qualitative aspects of a patient's status. For example, the arrow shown in FIG. 10 is partially filled, or colored, representing the relative progress that Mr. Jones has made relative to the normalized course of treatment. An arrow that is empty or white is indicative of little or no progress for a patient, and is thus appropriate for a patient that has just been diagnosed or just undergone treatment. Conversely, an arrow that is filled or fully colored is indicative of a patient that has completed his or her normalized course of therapy, and is thus ready for discharge. While an arrow is shown in FIG. 10 as representing a qualitative measure of a patient's status, other suitable shapes, colors, mnemonics, charts, graphs or devices could be readily used to convey the status of the patient in a qualitative fashion.

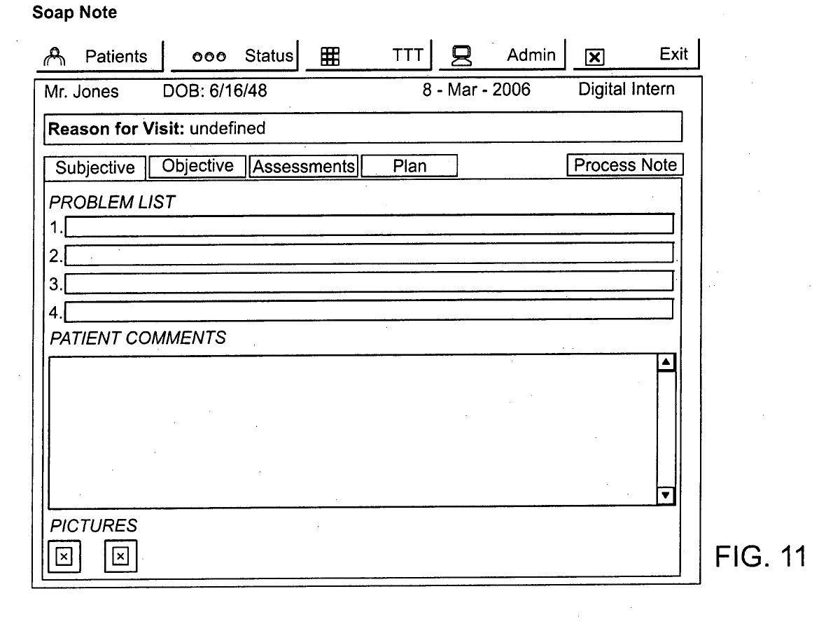

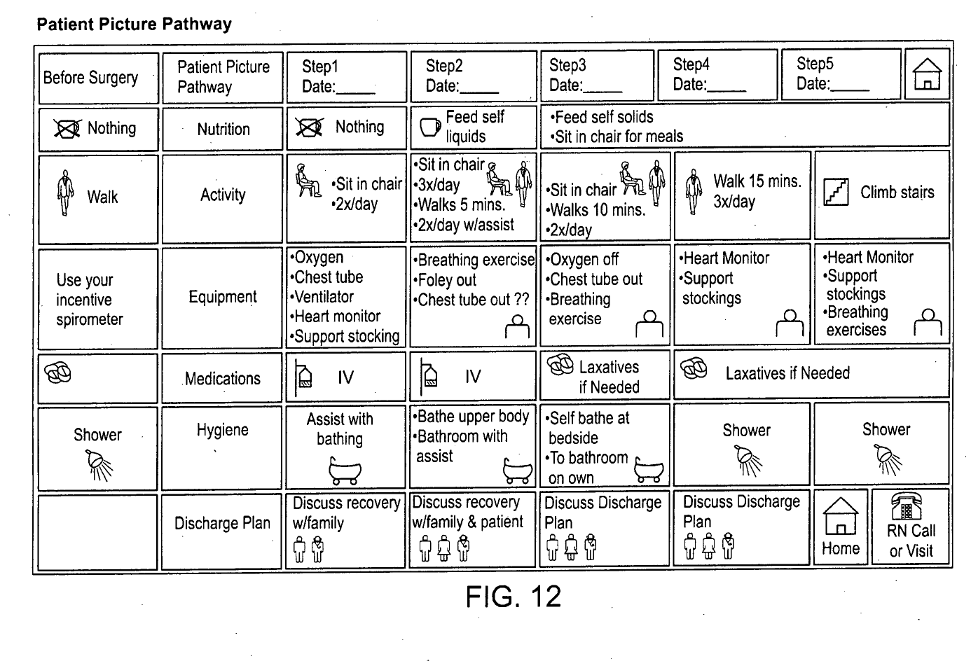

Other aspects and features of the present invention are shown in FIGS. 11 and 12, which are samples of the display 30 of the system 10. FIG. 11 is illustrative of a note entry field, which is selectable from the menu shown in FIG. 7. The note entry field functions too allow a user to enter various problems, patient comments and the like during the course of diagnosis and treatment. The note entry field includes a submenu, which in turn includes different tabs for notes related to subjective observations, objective observations, assessments and an associated plan for treatment. FIG. 12 is illustrative of a potential course of therapy for a selected patient and a selected disease process. Referred to as a patient picture pathway, the matrix of FIG. 12 presents a comprehensive treatment and path to recovery for a patient suffering from a selected disease process. For example, a surgical patient may have a recovery plan that includes aspects of nutrition, activity, equipment, medications, and hygiene, each of which can be monitored by the user. Progress can be planned and logged by the user as the patient moves through the defined steps to recovery and eventual discharge. Although described herein with reference to a surgical event, the matrix of FIG. 12 is equally applicable to other disease processes and normalized courses of therapy as defined herein.

As a person skilled in the art of information technology will recognize from the previous detailed description and from the figures and claims, modifications and changes can be made to the preferred embodiments of the invention without departing from the scope of this invention defined in the following claims.

We claim:

1. A method for representing patient data comprising:
(a) inputting clinical data regarding a patient into a selection algorithm;
(b) selecting actionable data from the clinical data; and
(c) representing a status of the patient in response to the actionable data.

2. The method of claim 1 further comprising the step of: (d) receiving the clinical data regarding the patient.

3. The method of claim 1 further comprising the step of: (e) defining a disease process for the patient.

4. The method of claim 3 further comprising the step of: (f) defining a normalized course of therapy for the disease process.

5. The method of claim 4 further comprising the step of: (g) comparing the actionable data and the normalized course for the patient and the disease process.

6. The method of claim 5 wherein step (c) includes visually presenting the patient status relative to the normalized course for the patient and the disease process.

7. The method of claim 6 wherein step (c) includes the step of defining a quantitative normalized course.

8. The method of claim 7 wherein step (c) further includes the step of presenting the patient status as a recovery score.

9. The method of claim 6 wherein step (c) includes the step of defining a qualitative normalized course.

10. The method of claim 9 wherein step (c) further includes the step of presenting the patient status as a milestone.

11. The method of claim 6 wherein step (c) further includes the step of providing access to the actionable clinical data.

12. The method of claim 8 further comprising the step of: (h) prompting a physician in response to an insufficient recovery score.

13. The method of claim 10 further comprising the step of: (i) prompting a physician in response to an insufficient milestone.

14. The method of claim 6 wherein step (c) further includes the step of providing access to non-actionable clinical data.

15. A system for representing patient data comprising:
a central computer including a database of patient data and a representation unit coupled to the database of patient data, wherein the representation unit includes a selection algorithm adapted to select between actionable and non-actionable data and a presentation unit adapted to format the actionable data into a presentable format; and
a mobile device communicable with the central computer, the mobile device including a display for displaying actionable data receivable in the presentable format from the central computer.

16. The system of claim 15 wherein the mobile device is communicable with the central computer through wireless means.

17. The system of claim 15 wherein the presentation unit is further adapted to format the non-actionable data in a presentable format.

18. The system of claim 15 wherein the database of patient data includes data regarding a diagnosis of a disease for each patient and further includes a disease process for each disease corresponding to each patient.

19. The system of claim 18 wherein the database of patient data includes data regarding a normalized course of therapy for each disease process.

20. The system of claim 19 wherein the selection algorithm is adapted to select between actionable and non-actionable data for each patient in response to the disease process and the normalized course.

21. The system of claim 20 wherein the presentation unit is adapted to format the actionable data into a presentable format in response to the disease process and the normalized course.

22. The system of claim 21 wherein the presentable format includes a quantitative representation of the patient status as a recovery score relative to the normalized course.

23. The system of claim 21 wherein the presentable format includes a qualitative representation of the patient status as a milestone relative to the normalized course.

24. The system of claim 21 wherein the presentation unit is further adapted to prompt a user of the mobile device in response to a deviation between the patient status and the normalized course.

25. The system of claim 15 further comprising a second mobile device communicable with the central computer.

26. The system of claim 25 wherein the second mobile device is communicable with the central computer through wireless means.

27. A system for representing patient data comprising:
a central computer including a database of patient data and means for representing patient data coupled to the database of patient data, wherein the means for representing patient data includes selection means for selecting between actionable and non-actionable data and presentation means for presenting the actionable data into a presentable format; and
a mobile device communicable with the central computer, the mobile device including a display for displaying actionable data receivable in the presentable format from the central computer.

28. The system of claim 27 wherein the mobile device is communicable with the central computer through wireless means.

29. The system of claim 27 wherein the presentation means includes means for formatting the non-actionable data in a presentable format.

30. The system of claim 27 wherein the database of patient data includes data regarding a diagnosis of a disease for each patient and further includes a disease process for each disease corresponding to each patient.

31. The system of claim 30 wherein the database of patient data includes data regarding a normalized course of therapy for each disease process.

32. The system of claim 31 wherein the selection means selects between actionable and non-actionable data for each patient in response to the disease process and the normalized course.

33. The system of claim 32 wherein the presentation means formats the actionable data into a presentable format in response to the disease process and the normalized course.

34. The system of claim 33 wherein the presentable format includes a quantitative representation of the patient status as a recovery score relative to the normalized course.

35. The system of claim 33 wherein the presentable format includes a qualitative representation of the patient status as a milestone relative to the normalized course.

36. The system of claim 33 further including means for prompting a user of the mobile device in response to a deviation between the patient status and the normalized course.

37. The system of claim 27 further comprising a second mobile device communicable with the central computer.

38. The system of claim 37 wherein the second mobile device is communicable with the central computer through wireless means.
